(19) United States
(12) Patent Application Publication
(10) Pub. No.: US 2011/0217751 A1
(43) Pub. Date: Sep. 8, 2011

(54) Alginate microbeads, method for forming the same and associated applications

(75) Inventors: Gwo-Bin Lee, Tainan City (TW); Song-Bin Huang, Tainan City (TW); Min-Hsien Wu, Kaohsiung City (TW)

(73) Assignee: National Cheng Kung University, Tainan City (TW)

(21) Appl. No.: 13/064,006

(22) Filed: Mar. 2, 2011

(30) Foreign Application Priority Data
Mar. 8, 2010 (TW) 099106581

(51) Int. Cl. C12N 11/10; C08B 37/04; H01F 1/00; B82Y 30/00

(52) U.S. Cl. 435/178; 536/3; 252/62.51 R; 977/700

(57) ABSTRACT

This present invention relates to a method for forming alginate microbeads. The method includes using a needle connected to a vibrator to continuously spot tiny alginate microdroplets in an oil layer. Subsequently, the temporarily formed alginate microdroplets sink into a CaCl₂ solution to become gelled microbeads. As a whole, the method has opened up a route to perform alginate-microbead formation in a simple, continuous, controllable, uniform, cell-friendly, and less-contaminated manner.

### BACKGROUND OF THE INVENTION

1. Field of the Invention

The present invention relates to a method for forming alginate microbeads and, more particularly, to a method for rapidly forming uniform alginate microbeads in a considerably reduced amount of oil/organic solutions.

2. Description of Related Art

Alginate microbeads (micrometer-scaled) have advantages such as high biocompatibility and ease of being manufactured, and thus have been evidenced that are applicable to drug-releasing treatment, scaffolds for encompassing cells and tissues, tissue and organ regeneration, and other biotechnical and medical uses.

Nowadays, most of the alginate microbeads are produced by three common methods. These common methods of forming alginate microbeads include emulsification, electro-spraying, and molding.

Emulsion for forming alginate microbeads is completed through following steps. First, a sodium alginate solution mixed with magnetic fluid is prepared as an aqueous reagent. Then, the aqueous reagent is mixed in an oil reagent and a proper surfactant is added therein. After the mixture is violently vibrated with an ultra-sonicator or agitator, emulsification occurs in the mixture and subsequently, a solidifying agent (CaCl₂/MgCl₂) is introduced therein for solidification. Finally, centrifugation, surface washing, and drying are carried out to collect solidified alginate microbeads.

However, emulsification requires considerable oil/organic solution, and the oil/organic solution will contaminate and influence applications of the formed alginate microbeads. Furthermore, although emulsified drops in a diameter of about 10 μm can be formed with a large-scale shaker, the uniformity of the emulsified alginate particles formed thereby cannot be accurately controlled and the particle size distribution thereof is limited in a range.

On the other hand, electro-spraying for forming alginate microbeads is completed through spraying out alginate solution in an electro field for molding. However, this method not only possibly damages the bio-sample but also produces particles with various sizes.

Moreover, molding for forming alginate microbeads is completed through following steps. First, a sodium alginate solution mixed with magnetic fluid is prepared. The sodium alginate solution is evenly mixed with magnetic fluid by an ultra-sonicator or agitator for a period of time that is based on the concentration of the alginate solution and the surface property of the magnetic fluid (i.e. the miscibility to sodium alginate). Direct solidification is conducted by injecting the mixed sample with a syringe to a CaCl₂/MgCl₂ solution. Reacting the sodium alginate solution with calcium ions forms solid calcium alginate, and thus magnetic beads where alginate encompasses magnetic particles can be directly obtained. However, the size of the alginate particles injected by a syringe is limited to that of the syringe, and it is limited in a range from 100 μm to 1 mm. Therefore, the magnetic beads are also of the limited size and not formed with a smaller size.

### SUMMARY OF THE INVENTION

In view of that various methods for forming alginate microbeads have their drawbacks, a considerable effort has been made by the inventors of the present invention for a long while to find out a novel method for forming alginate microbeads.

The object of the present invention is to provide a method for rapidly forming uniform alginate microbeads in a considerably reduced amount of oil/organic solutions.

To achieve the object, the method for forming alginate microbeads in the present invention comprises:

providing a treatment solution and a conveyer tube, wherein the treatment solution comprises an aqueous lower layer and an water-immiscible upper layer, and the conveyer tube has alginate therein and an outlet at one end thereof;

moving the alginate in the conveyer tube towards the outlet of the conveyer tube and suspending an alginate microdroplet at the outlet;

making the outlet at which the alginate microdroplet is suspended be inserted into or contact the water-immiscible upper layer of the treatment solution for a predetermined period of time, and then removing the outlet from the water-immiscible upper layer to retain the alginate microdroplet in the water-immiscible upper layer, wherein the alginate microdroplet instantly moves towards the aqueous lower layer; and

sinking the alginate microdroplet in the aqueous lower layer and to form a solidified alginate microbead.

In the method, suspending the alginate microdroplet, removing the outlet to retain the alginate microdroplet in the water-immiscible upper layer, and forming the solidified alginate microdroplet are repeated to form a plurality of alginate microbeads.

In the method, the aqueous lower layer can comprise a calcium chloride solution or a magnesium chloride solution. The water-immiscible upper layer can be any solution as long as the solution is immiscible to the aqueous lower layer, and it can comprise oil or an organic solution, for example, corn oil, sunflower oil, soybean oil, and n-hexadecane.

In the method, the aqueous lower layer can consist of a calcium chloride solution or a magnesium chloride solution, and the water-immiscible upper layer can consist of oil or an organic solution.

Preferably, the other end of the conveyer tube different from the outlet can be connected to a feeder capable of continuously supplying alginate.

Most preferably, the conveyer tube is a syringe, and the outlet is a needle of a syringe.

The present invention also relates to alginate microbeads, which are formed by the method mentioned above.

The present invention also relates alginate magnetic beads, which are formed by the method mentioned above, wherein magnetic nano-powder is mixed in the alginate.

The present invention also relates to an application of an alginate microbead used as a scaffold for 3D cell culture, wherein the alginate microbead is made by the method as claimed in claim 1 and cells are mixed in the alginate.

In the present invention, the drawback of conventionally requiring a large amount of oil or an organic solution in the emulsification to make microdrops can be overcome so as to reduce opportunity of contaminating the alginate microbeads during operation. Furthermore, because the oil/organic solution is required in a considerably reduced amount, alginate microdroplets in the oil/organic solution can rapidly sink in the lower CaCl₂/MgCl₂ solution based on the property of the alginate having larger density than that of the oil/organic solution and form uniform alginate microbeads.

In conclusion, the present invention can exhibit the following advantages:

(1) Compared with the emulsification, considerable reduction of the amount of the oil/organic solution can prevent the oil/organic solution from contaminating the formed alginate microbeads and influencing the applications thereof.

(2) Because of considerable reduction of the amount of the oil/organic solution, alginate microdroplets in the oil/organic solution can rapidly sink in the lower CaCl₂/MgCl₂ solution based on the property of the alginate having larger density than that of the oil/organic solution and then form uniform solidified alginate microbeads. Therefore, the problem of difficultly collecting solidified alginate microbeads formed in the emulsification can be overcome.

(3) The size of the formed alginate microbeads can be easily controlled.

Other objects, advantages, and novel features of the invention will become more apparent from the following detailed description when taken in conjunction with the accompanying drawings.

### BRIEF DESCRIPTION OF THE DRAWINGS

FIG. 1 shows a flow chart of the present invention;

FIG. 2 shows side views of forming alginate microdroplets in the present invention;

FIG. 3 is a histogram showing the relation among the flow rate of the alginate moving towards the needle, the frequency of the vertical vibration of the needle, and the diameter of the alginate microbeads in the present invention;

FIG. 4 shows microscopic pictures of the alginate in the present invention;

FIG. 5A is a microscopic picture showing an application of the alginate microbeads used as a scaffold for 3D cell culture in the present invention;

FIG. 5B is a fluorescence picture showing an application of the alginate microbeads used as a scaffold for 3D cell culture in the present invention; and

FIG. 6 is a microscopic picture showing the use of the alginate microbeads as the alginate magnetic beads in the present invention.

### DETAILED DESCRIPTION OF THE PREFERRED EMBODIMENT

The present invention is a novel method for forming alginate microbeads. After the alginate is transmitted to the needle, the needle is reciprocated in the oil/organic solution to form alginate microdroplets. The alginate microdroplets released from the needle rapidly sink in the lower CaCl₂/MgCl₂ solution and are reacted with the CaCl₂/MgCl₂ solution to form solidified alginate microbeads.

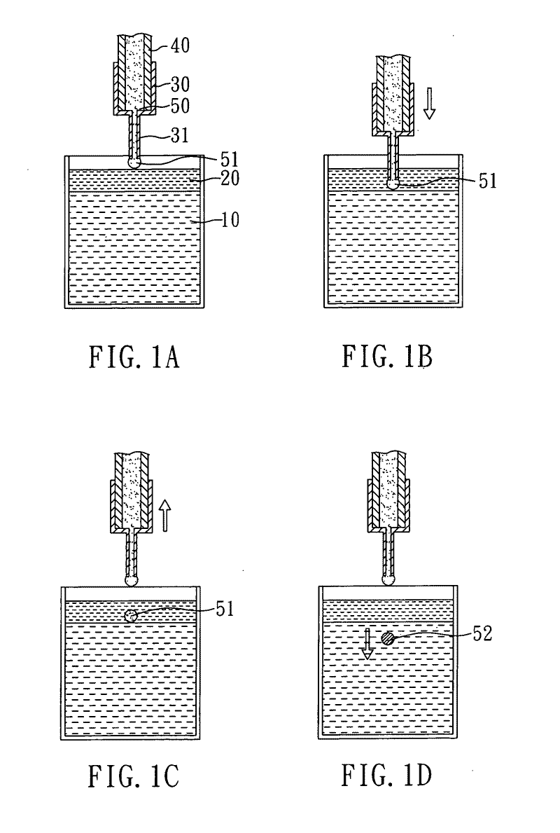

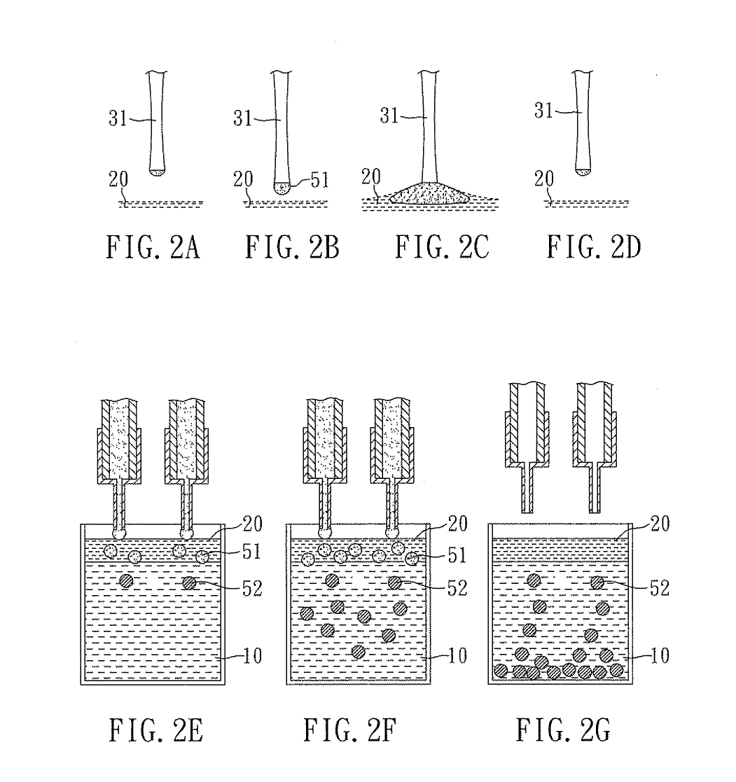

With reference to FIGS. 1 and 2, the method for forming alginate microbeads in the present invention includes the following steps.

As shown in FIG. 1A, a treatment solution and a conveyer tube are provided. The treatment solution includes a lower CaCl₂/MgCl₂ layer (10) and an upper oil/organic solution layer (20). In the present example, the conveyer tube is a syringe (30) and it is connected to a feeder (40) capable of supplying alginate (50).

As shown in FIGS. 1B, 2A, and 2B, an alginate microdroplet is suspended. This step includes continuously transmitting the alginate (50) towards the end of the needle (31) of the syringe (30) and suspending the alginate microdroplet (51) at the end of the needle (31).

As shown in FIGS. 1C, 2C, and 2D, the alginate microdroplet is released. This process includes the following steps. The needle (31) at which the alginate microdroplet (51) is suspended is inserted downward into or contacts the upper oil/organic solution layer (20) so that the alginate microdroplet (51) is immerged in the upper oil/organic solution layer (20). Then, the needle (31) is removed upwards from the upper oil/organic solution layer (20) so that the alginate microdroplet (51) is retained in the upper oil/organic solution layer (20). Based on the property of the alginate microdroplet (51) having larger density than that of the oil/organic solution, the alginate microdroplet instantly moves towards the lower CaCl₂/MgCl₂ layer (10).

As shown in FIGS. 1D and 2E, the alginate microdroplet is solidified. The alginate microdroplet (51) in the upper oil/organic solution layer (20) rapidly sinks in the lower CaCl₂/MgCl₂ layer (10) and forms a uniform solidified alginate microbead (52).

As shown in FIGS. 2F and 2G, the steps of suspending the alginate microdroplet, releasing the alginate microdroplet, and solidifying the alginate microdroplet are repeated to form a plurality of the alginate microbeads.

In the method, the predetermined period of time refers to the frequency of the vertical vibration of the needle. Because the frequency of the vertical vibration of the needle and the flow rate of supplying the alginate are both related to the desired size of the alginate microbeads, one skilled in the art can modify those parameters based on the desired size of the alginate microbeads. Hence, the predetermined period of time and the flow rate of supplying the alginate are not limited in a specific value.

Although the rate of producing the alginate microbeads in the formation of the present invention carried out once is smaller than that in the emulsification, the present invention can substantially improve the rate of producing the alginate microbeads by an array (i.e., the alginate is transmitted once in an array of needles, and then the array of the needles follows the method mentioned above).

### EXAMPLE

The following example are only used to illustrate the present invention and to make one skilled in the art understand and carry out the present invention, but not to restrict the scope of the present invention in any manner. Therefore, any modification and change in accordance with the concept of the present invention is still in the scope of the present invention.

### I. The Relation Among the Flow Rate of the Alginate Moving Towards the Needle, the Frequency of the Vertical Vibration of the Needle, And the Diameter of the Alginate Microbeads

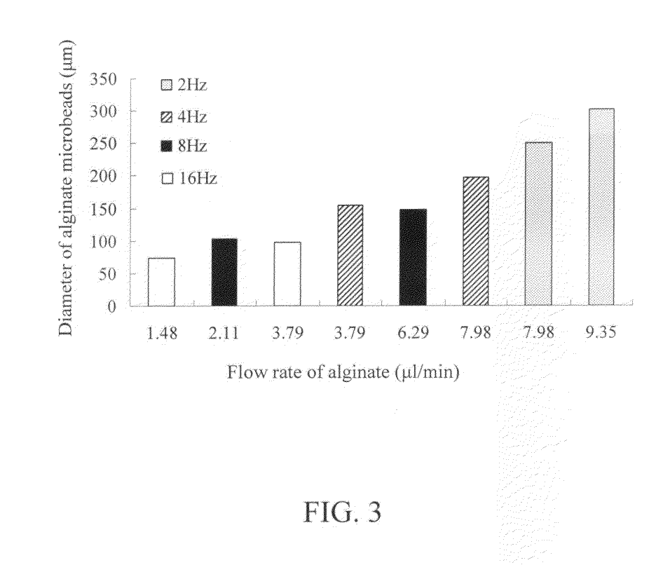

With reference to FIG. 3, when the frequency of the vertical vibration of the needle is 2 Hz, the rate of supplying the alginate is controlled at 7.98 μL/min and 9.35 μL/min. The diameters of the alginate microbeads are about 255 μm and 302 μm, respectively. When the frequency of the vertical vibration of the needle is 4 Hz, the rate of supplying the alginate is controlled at 3.79 μL/min and 7.98 μL/min. The diameters of the alginate microbeads are about 160 μm and 200 μm, respectively. When the frequency of the vertical vibration of the needle is 8Hz, the rate of supplying the alginate is controlled at 2.11 μL/min and 6.29 μL/min. The diameters of the alginate microbeads are about 110 μm and 148 μm, respectively.

When the frequency of the vertical vibration of the needle is 16 Hz, the rate of supplying the alginate is controlled at 1.48 μL/min and 3.79 μL/min. The diameters of the alginate microbeads are about 72 μm and 100 μm, respectively.

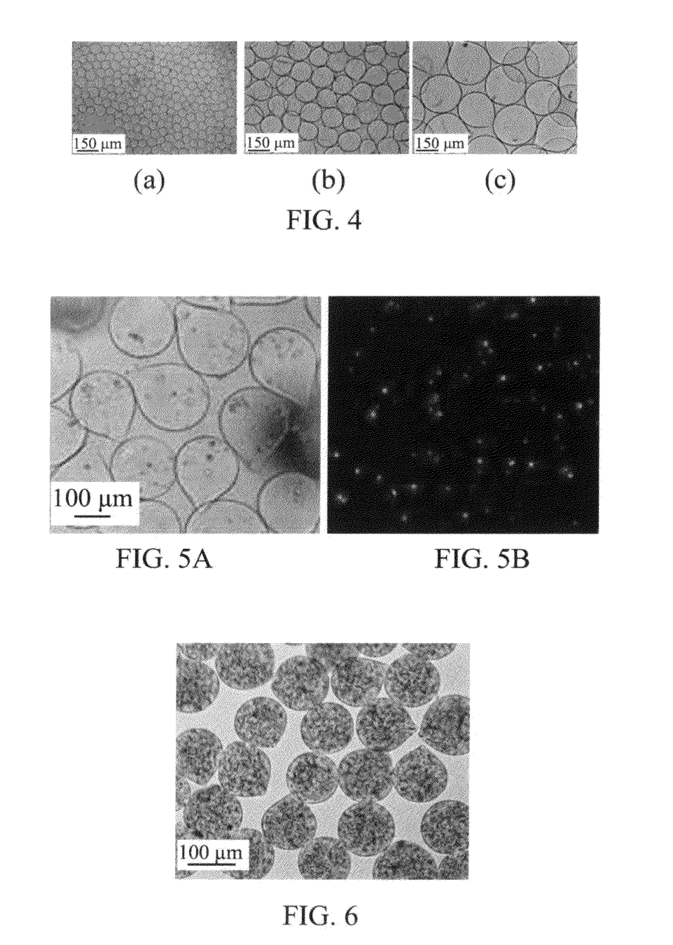

FIG. 4 (a) shows the alginate microbeads formed when the frequency of the vertical vibration of the needle is 16 Hz and the rate of supplying the alginate is controlled at 1.48 μL/min. FIG. 4 (b) shows the alginate microbeads formed when the frequency of the vertical vibration of the needle is 8 Hz and the rate of supplying the alginate is controlled at 6.29 μL/min. FIG. 4 (b) shows the alginate microbeads formed when the frequency of the vertical vibration of the needle is 2 Hz and the rate of supplying the alginate is controlled at 9.35 μL/min. The microscope used in the observations mentioned above is TE300 (Nikon, USA).

Different combinations between the flow rate of supplying the alginate and the frequency of the vertical vibration of the needle can make the alginate microbeads in various sizes. Hence, the method of the present invention can form the alginate microbeads in various sizes based on the needs.

### II. The Application of the Alginate Microbeads Used As A Scaffold For 3D Cell Culture

Cells are mixed in the alginate. The alginate microbeads encompassing the cells are formed according the method of the present invention. Thus, the 3D cell culture can be accomplished. FIG. 5A shows a view of the light field taken by a microscope. FIG. 5B is a fluorescence picture where green spots represent live cells and red spots represent death cells. Accordingly, it can be seen that the method of the present invention is suitable to encompass cells and will not injure live cells. In the observations mentioned above, the microscope used is TE300 (Nikon, USA). The survivability of the cells is determined by a fluorescent dye kit which is LIVE/DEADViability/Cytotoxicity Kit L-3224 and the molecular probes are used.

### III. The Application of the Alginate Microbeads As the Alginate Magnetic Beads

Magnetic nano-powder is mixed in the alginate. The alginate magnetic beads encompassing the magnetic nano-powder are formed according the method of the present invention, as shown in FIG. 6. The alginate magnetic beads can be used to replace commercial magnetic beads at present and applied in an important step of the purification of the sample in biomedical clinical diagnosis requiring magnetic beads.

The present invention not only overcomes the drawback in the conventional emulsification of producing microdroplets in need of considerable oil or organic solution, but also reduces the opportunity of the alginate microbeads being contaminated during operation. Therefore, uniform and size-controllable alginate microbeads can be formed.

The alginate microbeads of the present invention can be used as a scaffold for 3D cell culture and also as alginate magnetic beads. Hence, the present invention has remarkable contribution to the related industries.

Although the present invention has been explained in relation to its preferred embodiment, it is to be understood that many other possible modifications and variations can be made without departing from the scope of the invention as hereinafter claimed.

1. A method for forming an alginate microbead, comprising:
providing a treatment solution and a conveyer tube, wherein the treatment solution comprises an aqueous lower layer and an water-immiscible upper layer, and the conveyer tube has alginate therein and an outlet at one end thereof;
moving the alginate in the conveyer tube towards the outlet of the conveyer tube and suspending an alginate microdroplet at the outlet;
making the outlet at which the alginate microdroplet is suspended be inserted into or contact the water-immiscible upper layer of the treatment solution for a predetermined period of time, and then removing the outlet from the water-immiscible upper layer to retain the alginate microdroplet in the water-immiscible upper layer, wherein the alginate microdroplet instantly moves towards the aqueous lower layer; and
sinking the alginate microdroplet in the aqueous lower layer to form a solidified alginate microbead.

2. The method as claimed in claim 1, wherein the aqueous lower layer comprises a calcium chloride solution or a magnesium chloride solution, and the water-immiscible upper layer comprises oil or an organic solution.

3. The method as claimed in claim 1, wherein the aqueous lower layer consists of a calcium chloride solution or a magnesium chloride solution, and the water-immiscible upper layer consists of oil or an organic solution.

4. The method as claimed in claim 1, wherein the other end of the conveyer tube different from the outlet is connected to a feeder capable of continuously supplying alginate.

5. The method as claimed in claim 4, wherein the conveyer tube is a syringe, and the outlet is a needle of a syringe.

6. The method as claimed in claim 1, wherein suspending the alginate microdroplet, removing the outlet to retain the alginate microdroplet in the water-immiscible upper layer, and forming the solidified alginate microdroplet are repeated to form a plurality of alginate microbeads.

7. The method as claimed in claim 4, wherein suspending the alginate microdroplet, removing the outlet to retain the alginate microdroplet in the water-immiscible upper layer, and forming the solidified alginate microdroplet are repeated to form a plurality of alginate microbeads.

8. The method as claimed in claim 5, wherein suspending the alginate microdroplet, removing the outlet to retain the alginate microdroplet in the water-immiscible upper layer, and forming the solidified alginate microdroplet are repeated to form a plurality of alginate microbeads.

9. An alginate microbead, which is formed by the method as claimed in claim 1.

10. An alginate magnetic bead, which is formed by the method as claimed in claim 1, wherein magnetic nano-powder is mixed in the alginate.

11. An application of an alginate microbead used as a scaffold for 3D cell culture, wherein the alginate microbead is made by the method as claimed in claim 1 and cells are mixed in the alginate.
